(19) United States
(12) Patent Application Publication
(10) Pub. No.: US 2025/0333392 A1
(43) Pub. Date: Oct. 30, 2025

(54) SYNTHESIS OF (-)-TRANS-DELTA-9-TETRAHYDROCANNABIVARIN (DELTA-9 THCV) AND ANALOGS THEREOF

(71) Applicant: NALU BIO, INC., San Francisco, CA (US)

(72) Inventors: Mehdi Haghdoost, Stittsville (CA); Matthew Roberts, Calabasas, CA (US); Brian Thomas, Manteo, NC (US); Luke Dickinson, Citrus Heights, CA (US); Edward Nikitin, Durham, NC (US)

(21) Appl. No.: 19/173,695

(22) Filed: Apr. 8, 2025

Related U.S. Application Data
(60) Provisional application No. 63/631,401, filed on Apr. 8, 2024.
(60) Provisional application No. 63/640,638, filed on Apr. 30, 2024.

(51) Int. Cl. C07D 311/80; C07D 313/20

(52) U.S. Cl. CPC C07D 311/80; C07D 313/20

(57) ABSTRACT

A method is provided for the synthesis of (-)-trans-Δ⁹-tetrahydrocannabivarin (Δ⁹-THCV) and analogs thereof such that in the reaction product, the molar ratio of the Δ⁹ isomer to incidentally formed Δ⁸ isomers is greater than 4:1. Synthesis is carried out by combining a selected cannabinoid reactant, e.g., cannabidivarin (CBDV) or an analog thereof, with an acid in a solvent for the cannabinoid reactant, wherein the acid comprises (i) a Lewis acid having an acid softness index value in the range of −10 ΔG⁰_{f, M}ⁿ⁺ to −150 ΔG⁰_{f, M}ⁿ⁺, (ii) a Brønsted acid having a pKa in the range of −4.0 to +4.0, or (iii) a combination of (i) and (ii), under reaction conditions comprising a reaction temperature in the range of −0° C. to 25° C. and a reaction time in the range of 1 hour to 24 hours. The reaction is thereafter quenched with base and the solvent removed, wherein the crude reaction product so provided may be purified, e.g., chromatographically purified. Also provided is a method for synthesizing Δ⁹-THCV that further includes synthesis of the cannabinoid reactant. The invention additionally provides novel cannabinoid compositions that may be synthesized using the aforementioned methodology.

### CROSS-REFERENCE TO RELATED APPLICATIONS

This application claims priority under 35 U.S.C. § 119(e) to provisional U.S. Patent Applications Ser. No. 63/631,401, filed Apr. 8, 2024, and 63/640,368, filed Apr. 30, 2024, the disclosures of which are incorporated by reference herein.

### TECHNICAL FIELD

The present invention relates generally to cannabinoids, and more particularly relates to a method for chemically synthesizing cannabinoids and cannabinoid analogs. The invention has utility in the fields of medicine, medicinal chemistry, therapeutics, and chemical and pharmaceutical manufacturing.

### BACKGROUND

Medical cannabis has received extensive attention in the media and the scientific literature. More recently, individual plant cannabinoids isolated from the Cannabis sativa (C. Sativa) plant have been researched and proposed for use in many medicinal contexts. These plant cannabinoids include compounds such as cannabidiol (CBD), cannabinol (CBN), cannabichromene (CBC), cannabidiolic acid (CBDA), tetrahydrocannabinolic acid (Δ⁹-THCA), tetrahydrocannabiphorol (THCP), (-)-trans-Δ⁹-tetrahydrocannabivarin (also referred to herein as Δ⁹-tetrahydrocannabivarin or Δ⁹-THCV), and tetrahydrocannabivarinic acid (THCVA), with CBD and Δ⁹-THCV of particular interest.

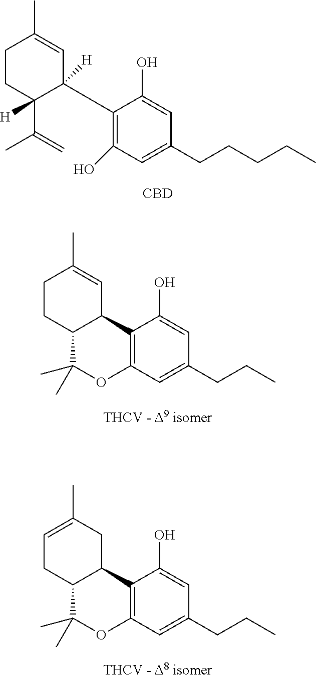

The potential medicinal properties of THCV and other similar cannabinoids are attributed to specific interaction with the CB₁ and CB₂ receptors as well as many other receptors of the endocannabinoid system. These receptors are located in the brain and throughout the central and peripheral nervous systems. Activation of the CB₁ receptors, in particular, leads to inhibition of adenylyl cyclase activity and blockade of voltage-operated calcium channels, thereby suppressing neuronal excitability and serotonin neurotransmission inhibition. As a result, it has been suggested that cannabinoids that activate CB₁ receptors have potential utility in treatment of depression, neurological diseases, chronic pain, multiple sclerosis, glaucoma, and other conditions. See, e.g., Abioye et al. (2020), “Δ⁹-tetrahydrocannabivarin: a commentary on potential therapeutic benefit for the management of obesity and diabetes,” J. Cannabis Res. 2:1-6.

THCV is a neutral CB₁ antagonist that has also been proposed as a therapeutic compound for the treatment of obesity and obesity-associated metabolic disorders such as type 2 diabetes and glucose intolerance (Kowalczuk et al. (2023), “Tetrahydrocannabivarin (THCV) Protects Adipose-Derived Mesenchymal Stem Cells (ASC) against Endoplasmic Reticulum Stress Development and Reduces Inflammation during Adipogenesis,” Int J Mol Sci. 24(8): 1-21; Wargent et al. (2013), “The cannabinoid compound Δ⁹-tetrahydrocannabivarin (THCV) ameliorates insulin sensitivity in two mouse models of obesity,” Nutr. Diabetes 3(5): e68; Jadoon et al. (2016), “Efficacy and Safety of Cannabidiol and Tetrahydrocannabivarin on Glycemic and Lipid Parameters in Patients with Type 2 Diabetes . . . ,” Diabetes Care 39(10): 1777-86

THCV in the C. sativa plant primarily occurs as the (-)-trans-Δ⁸-THCV (“Δ⁸-THCV”) and (-)-trans-Δ⁹-THCV (“Δ⁹-THCV”) isomers (molecular structures shown above), with the Δ⁸ isomer a considerably weaker CB₁ antagonist than the Δ⁹ isomer. As recently reported, Δ⁸-THCV has about two times lower potency than the Δ⁹ counterpart; see Walsh and Holmes (2022), “Pharmacology of Minor Cannabinoids at the Cannabinoid CB₁ Receptor: Isomer-and Ligand-Dependent Antagonism by Tetrahydrocannabivarin,” Receptors1(1): 3-12. While the aforementioned THCV isomers can be extracted from the C. sativa plant, methods for chemically synthesizing THCV have also been attempted. These tend to result in a reaction product composition having a significant proportion of the less desirable Δ⁸ isomer, however, and separation of the two isomers is required to obtain pure Δ⁹-THCV.

Accordingly, there is a need in the art for a method to preferentially synthesize the Δ⁹-THCV isomer, wherein an ideal method would, among other desired advantages, provide the desired product in high yield, involve a minimal number of steps, use environmentally benign reagents and solvents, and be readily scaled up to provide a viable manufacturing process.

### SUMMARY OF THE INVENTION

The invention is directed to the above-mentioned need in the art and provides a method for synthesizing Δ⁹-THCV and analogs thereof, wherein the method provides numerous advantages relative to prior known methods of synthesizing Δ⁹-THCV.

The novel method employs cannabidivarin (CBDV) or an analog thereof as a cannabinoid reactant and provides a reaction product composition in which the desired Δ⁹ isomer is predominant. The method is simple and straightforward without necessitating multiple synthetic steps; may be carried out under mild reaction conditions; may be implemented at large scale in the manufacturing context; and is characterized by rapid reaction rate and low cost. In addition, the method may be readily tailored to synthesize analogs of Δ⁹-THCV as well as Δ⁹-THCV per se by using appropriately substituted reactants as will be described in detail herein.

In a first embodiment, then, a method is provided for synthesizing a Δ⁹ cannabinoid having the structure of formula (II)

wherein:

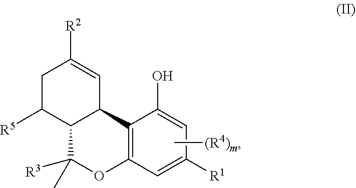

R¹ is selected from C₁-C₁₂ hydrocarbyl, substituted C₁-C₁₂ hydrocarbyl, heteroatom-containing C₁-C₁₂ hydrocarbyl, and substituted heteroatom-containing C₁-C₁₂ hydrocarbyl;

R², R³, and R⁵ are independently selected from C₁-C₆ alkyl and substituted C₁-C₆ alkyl;

m is zero, 1 or 2; and

R⁴ is OH or OR⁶ wherein R⁶ is H, C₁-C₆ alkyl, C₅-C₁₂ aryl, or a hydroxyl protecting group, with the proviso that when m is 2, the R⁴ may be the same or different,

wherein the method comprises:
(a) combining a cannabinoid reactant having the structure of formula (I)

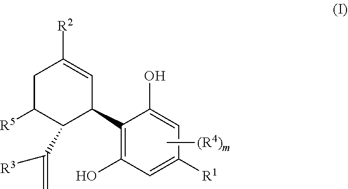

with an acid in a solvent for the cannabinoid reactant and the acid to provide a reaction mixture, wherein the acid comprises (i) a Lewis acid having an acid softness index value in the range of −10 ΔG⁰_{f, M}ⁿ⁺ to −150 ΔG⁰_{f, M}ⁿ⁺, (ii) a Brønsted acid having a pKa in the range of −4.0 to +4.0, or (iii) a combination of (i) and (ii), under reaction conditions comprising a reaction temperature in the range of −0° C. to 25° C. and a reaction time in the range of 1 hour to 24 hours;
(b) quenching the reaction mixture of (a) with a base; and
(c) removing the solvent to provide a reaction product comprising the Δ⁹ cannabinoid having the structure of formula (II).

In one embodiment:
R¹ is selected from C₁-C₈ alkyl, substituted C₁-C₈ alkyl, heteroatom-containing C₁-C₈ alkyl, substituted heteroatom-containing C₁-C₈ alkyl, C₂-C₈ alkenyl, substituted C₂-C₈ alkenyl, heteroatom-containing C₂-C₈ alkenyl, and substituted heteroatom-containing C₂-C₈ alkenyl; R² and R³ are C₁-C₆ alkyl and may be the same or different;
R⁵ is H; and
m is zero.

In this embodiment, the reaction product comprises a mixture of cannabinoids, including, without limitation: the desired Δ⁹ isomer having the structure of formula (II); and Δ⁸ isomers thereof, the Δ⁸ isomers having the structures of formulae (II-A), (II-B), and (II-C)

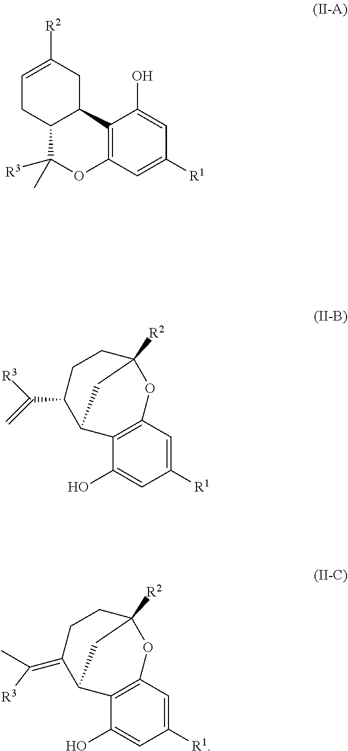

In some embodiments, the aforementioned mixture of cannabinoids in the reaction product comprises the Δ⁹ cannabinoid having the structure of formula (II) in a molar ratio, relative to the Δ⁸ isomers of formulae (II-A), (II-B), and (II-C), of greater than 4:1.

In other embodiments, the molar ratio of the Δ⁹ cannabinoid to the total of the Δ⁸ isomers of formulae (II-A), (II-B), and (II-C) in the reaction product is in the range of 4:1 to 50:1.

In other embodiments, the molar ratio of the Δ⁹ cannabinoid to the total of the Δ⁸ isomers of formulae (II-A), (II-B), and (II-C) in the reaction product is in the range of 9:1 to 18:1.

The reaction product may be purified to substantially increase the aforementioned molar ratio. In some embodiments, a chromatographic purification process is used, for instance using normal phase silica column chromatography and an isocratic mobile phase. In the purified reaction product so obtained, the Δ⁹ cannabinoid having the structure of formula (II) is in a molar ratio, relative to the Δ⁸ isomers of formulae (II-A), (II-B), and (II-C), of greater than 50:1. In other embodiments, the molar ratio of the Δ⁹ cannabinoid to the Δ⁸ isomers in the purified reaction product is in the range of 50:1 to 1000:1.

In some embodiments, the method of the invention employs as the acid a Lewis acid having an acid softness index value in the range of −10 ΔG⁰_{f, M}^{N+} to −150 ΔG⁰_{f, M}ⁿ⁺. The Lewis acid generally comprises a salt of a Group 13 element of the periodic table (also referred to as Group IIIB), a transition metal in the +2 oxidation state, a transition metal in the +3 oxidation state, an actinide, or a lanthanide.

In other embodiments, the method of the invention employs as the acid a Brønsted acid having a pKa in the range of −4.0 to +4.0. In some aspects of these embodiments, the Brønsted acid has a pKa in the range of −2.0 to +2.0.

In some embodiments of the present synthetic method, the concentration of the cannabinoid reactant in the solvent is in the range of 0.25 M to 2.5 M, for instance in the range of 0.25 M to 1.5 M.

In some embodiments of the present synthetic method, the acid is present in the reaction mixture at a molar ratio in the range of 0.01:1 to 0.2:1 relative to the cannabinoid reactant, including a molar ratio in the range of 0.05:1 to 0.1:1 and a molar ratio in the range of 0.07:1 to 0.1:1.

In some embodiments, R¹ in structures (I), (II), (II-A), (II-B), and (II-C) is an optionally substituted C₁-C₈ alkyl or C₂-C₈ alkenyl group.

In other embodiments, R¹ in structures (I), (II), (II-A), (II-B), and (II-C) is a C₂-C₆ alkyl group.

In some embodiments, In the structures of formulae (I), (II), (II-A), (II-B), and (II-C), R¹ is n-propyl, R² and R³ are methyl, m is zero, and R⁵ is H, such that the cannabinoid reactant having the structure of formula (I) is cannabidivarin and the Δ-9 cannabinoid having the structure of formula (II) is Δ⁹-tetrahydrocannabivarin. In these embodiments, the cannabidivarin used as the cannabinoid reactant may be derived from hemp. Alternatively, the cannabidivarin employed may be chemically synthesized using the methodology described in applicant's published international patent application WO 2022/133332 A2 and in co-pending U.S. patent application Ser. No. 18/212,061, filed Jun. 20, 2023 and published on Nov. 9, 2023 as US 2023/0357177 A1. The disclosures of the foregoing patent applications are incorporated by reference in their entireties.

In one embodiment, the cannabidivarin used as the cannabinoid reactant is synthesized by contacting divarinol with Δ⁹-2,8-menthadien-1-ol in the presence of a Lewis acid catalyst under reaction conditions effective to result in a reaction product comprising cannabidivarin.

In another embodiment, the cannabidivarin used as the cannabinoid reactant is synthesized from phloroglucinol by a method comprising: contacting the phloroglucinol with a hydroxyl-protecting reagent to provide hydroxyl-protected phloroglucinol; carrying out a cross-coupling reaction of the hydroxyl-protected phloroglucinol with a reactant M-CH₂CH₂CH₃ in the presence of a catalyst that facilitates the cross-coupling reaction, wherein M comprises a metallic element, to provide hydroxyl-protected divarinol; deprotecting the hydroxyl-protected divarinol; and contacting the divarinol with Δ⁹-2,8-menthadien-1-ol in the presence of a Lewis acid catalyst under reaction conditions effective to result in a reaction product comprising cannabidivarin.

The invention is also directed to a cannabinoid composition comprising Δ⁹-THCV, Δ⁸-THCV, Δ⁸-iso-THCV, Δ⁴⁽⁸⁾-iso-THCV, and a Lewis acid catalyst residue, wherein the molar ratio of the Δ⁹-THCV to the total of the Δ⁸-THCV, Δ⁸-iso-THCV, and Δ⁴⁽⁸⁾-iso-THCV is greater than 4:1 and the Lewis acid catalyst residue represents 1-150 ppm of the composition. In some embodiments, the Lewis acid catalyst residue comprises aluminum, resulting from the use of AlCl³ as the acid in the present synthetic method.

The invention further encompasses a purified cannabinoid composition comprising Δ⁹-THCV, Δ⁸-THCV, Δ⁸-iso-THCV, Δ⁴⁽⁸⁾-iso-THCV, and a Lewis acid catalyst residue, wherein the molar ratio of the Δ⁹-THCV to the total of the Δ⁸-THCV, Δ⁸-iso-THCV, and Δ⁴⁽⁸⁾-iso-THCV is greater than 50:1 and the Lewis acid catalyst residue represents 1-150 ppm of the composition. As in the preceding embodiment, the Lewis acid catalyst residue may comprise aluminum, again resulting from the use of AlCl ₃ as the acid in the present synthetic method.

### BRIEF DESCRIPTION OF THE DRAWINGS

FIG. 1 is a ¹H NMR spectrum of a reaction product generated using the synthetic method described in Examples 1-3.

FIG. 2 is a high-performance liquid chromatogram for an early eluting fraction of the purified reaction product of Example 4.

FIG. 3 is a high-performance liquid chromatogram for a late eluting fraction of the purified reaction product of Example 4.

FIG. 4 is a process flow diagram for the synthesis of Δ⁹-THCV as described in Example 6.

### DETAILED DESCRIPTION OF THE INVENTION

### I. Definitions and Nomenclature

### A. General

Unless defined otherwise, all technical and scientific terms used herein have the meaning commonly understood by one of ordinary skill in the art to which the invention pertains. Specific terminology of particular importance to the description of the present invention is defined below.

In this specification and the appended claims, the singular forms “a,” “an” and “the” include plural referents unless the context clearly dictates otherwise.

### B. Chemical Terminology

As used herein, the phrase “having the formula” or “having the structure” is not intended to be limiting and is used in the same way that the term “comprising” is used.

The term “hydrocarbyl” refers to hydrocarbyl groups or linkages containing 1 to about 18 carbon atoms, typically 1 to 12 carbon atoms, including linear, branched, cyclic, saturated, and unsaturated species, such as alkyl groups, alkenyl groups, aryl groups, and the like. “Substituted hydrocarbyl” refers to hydrocarbyl substituted with one or more substituent groups, and the term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

The term “alkyl” as used herein refers to a branched or unbranched saturated hydrocarbon group typically although not necessarily containing 1 to about 18 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl, and the like. Generally, although again not necessarily, alkyl groups herein contain 1 to 12 carbon atoms, e.g., 1 to 12 carbon atoms, 1 to 10 carbon atoms, 1 to 8 carbon atoms, 1 to 6 carbon atoms or 1 to 3 carbon atoms. “Substituted alkyl” refers to alkyl substituted with one or more substituent groups, and the terms “heteroatom-containing alkyl” and “heteroalkyl” refer to alkyl in which at least one carbon atom is replaced with a heteroatom, as described in further detail infra. If not otherwise indicated, the term “alkyl” includes linear, branched, cyclic, unsubstituted, substituted, and/or heteroatom-containing alkyl.

The term “alkenyl” as used herein refers to a linear, branched, or cyclic hydrocarbon group of 2 to about 18 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Alkenyl groups herein typically contain 2 to 12 carbon atoms, e.g., 2 to 12 carbon atoms, 2 to 10 carbon atoms, 2 to 8 carbon atoms, 2 to 6 carbon atoms or 2 to 3 carbon atoms. The term “cycloalkenyl” intends a cyclic alkenyl group, typically having 5 to 8 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the term “alkenyl” includes linear, branched, cyclic, unsubstituted, substituted, and/or heteroatom-containing alkenyl.

The term “alkynyl” as used herein refers to a linear or branched hydrocarbon group of 2 to 18 carbon atoms containing at least one triple bond, such as ethynyl, n-propynyl, and the like. Generally, although again not necessarily, alkynyl groups herein contain 2 to 12 carbon atoms, e.g., 2 to 12 carbon atoms, 2 to 10 carbon atoms, 2 to 8 carbon atoms, 2 to 6 carbon atoms or 2 to 3 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the term “alkynyl” includes linear, branched, unsubstituted, substituted, and/or heteroatom-containing alkynyl.

The term “alkoxy” as used herein refers to an alkyl group bound through a single, terminal ether linkage; that is, an “alkoxy” group may be represented as —O-alkyl where alkyl is as defined above. Alkoxy groups thus include C₁-C₆ alkoxy groups such as methoxy, ethoxy, n-propoxy, isopropoxy, t-butyloxy, etc. The terms “alkenyloxy” and “alkynyloxy” are defined in an analogous manner.

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 18 carbon atoms, and particularly preferred aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatic substituents.

The term “aryloxy” as used herein refers to an aryl group bound through a single, terminal ether linkage, wherein “aryl” is as defined above. An “aryloxy” group may be represented as —O-aryl where aryl is as defined above. Preferred aryloxy groups contain 5 to 18carbon atoms, and particularly preferred aryloxy groups contain 5 to 14 carbon atoms. Examples of aryloxy groups include, without limitation, phenoxy, o-halo-phenoxy, m-halo-phenoxy, p-halo-phenoxy, o-methoxy-phenoxy, m-methoxy-phenoxy, p-methoxy-phenoxy, 2,4-dimethoxy-phenoxy, 3,4,5-trimethoxy-phenoxy, and the like.

The term “alkaryl” refers to an aryl substituent that is substituted with an alkyl group, and the term “aralkyl” refers to an alkyl substituent that is substituted with an aryl group, wherein “aryl” and “alkyl” are as defined above. Preferred alkaryl and aralkyl groups contain 6 to 18 carbon atoms, and particularly preferred aralkyl groups contain 6 to 16 carbon atoms. For example, alkaryl groups include, for example, p-methylphenyl, 2,4-dimethylphenyl, p-cyclohexylphenyl, 2,7-dimethylnaphthyl, 7-cyclooctyinaphthyl, 3-ethyl-cyclopenta-1,4-diene, and the like. Aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. The terms “alkaryloxy” and “aralkyloxy” refer to substituents of the formula-OR wherein R is alkaryl or aralkyl, respectively, as just defined.

The term “acyl” refers to substituents having the formula —(CO)-alkyl, —(CO)-aryl, or —(CO)-aralkyl, and the term “acyloxy” refers to substituents having the formula —O(CO)-alkyl, —O(CO)-aryl, or —O(CO)-aralkyl, wherein “alkyl,” “aryl, and “aralkyl” are as defined above.

The term “cyclic” refers to alicyclic or aromatic substituents that may or may not be substituted and/or heteroatom containing, and that may be monocyclic, bicyclic, or polycyclic.

The term “alicyclic” is used in the conventional sense to refer to an aliphatic cyclic moiety, as opposed to an aromatic cyclic moiety, and may be monocyclic, bicyclic, polycyclic, and may be bridged.

The terms “halo” and “halogen” are used in the conventional sense to refer to a chloro, bromo, fluoro, or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage, or substituent in which one or more carbon atoms are replaced with an atom other than carbon, e.g., nitrogen, oxygen, sulfur, phosphorus, or silicon, typically nitrogen, oxygen, or sulfur, preferably nitrogen or oxygen. Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

By “substituted” as in “substituted alkyl,” “substituted aryl,” and the like, as alluded to in some of the aforementioned definitions, is meant that in the alkyl, aryl, or other moiety, at least one hydrogen atom bound to a carbon (or other) atom is replaced with one or more non-hydrogen substituents. Examples of such substituents include functional groups and hydrocarbyl moieties.

Functional groups that may represent substituents in the substituted molecular structures and segments thereof include, without limitation: halo, hydroxyl, sulfhydryl, C₁-C₁₈ alkoxy, C₂-C₁₈ alkoxyalkyl, C₂-C₁₈ alkenyloxy, C₂-C₁₈ alkynyloxy, C₅-C₁₈ aryloxy, acyl (including C₂-C₁₈ alkylcarbonyl (—CO-alkyl) and C₆-C₁₈ arylcarbonyl (—CO-aryl)), acyloxy (-O-acyl), C₂-C₁₈ alkoxycarbonyl (—(CO)-O-alkyl), C₆-C₁₈ aryloxycarbonyl (—(CO)—O-aryl), halocarbonyl (—CO)—X where X is halo), C₂-C₁₈ alkylcarbonato (—O—(CO)—O-alkyl), C₆-C₁₈ arylcarbonato (—O—(CO)—O-aryl), carboxy (—COOH), carboxylato (—COO—), carbamoyl (—(CO)—NH₂), mono-(C₁-C₁₈ alkyl)-substituted carbamoyl (-(CO)-NH (C₁-C₁₈ alkyl)), di-(C₁-C₁₈ alkyl)-substituted carbamoyl (-(CO)-N (C₁-C₁₈ alkyl)₂), mono-(C₅-C₁₈ aryl)-substituted carbamoyl (—(CO)—NH-aryl), di-(C₅-C₁₈ aryl)-substituted carbamoyl (—(CO)—N(aryl)₂), di-N-(C₁-C₁₈ alkyl), N-(C₅-C₁₈ aryl)-substituted carbamoyl, thiocarbamoyl (—(CS)—NH₂), carbamido (—NH—(CO)—NH₂), cyano (—C≡N), isocyano (—N⁺≡C⁻—), cyanato (—O—C≡N), isocyanato (—O—N⁺≡C⁻—), isothiocyanato (—S—C≡N), azido (—N═N⁺≡N⁻), formyl (—(CO)—H), thioformyl (—(CS)—H), amino (—NH₂), mono-(C₁-C₁₈ alkyl)-substituted amino, di-(C₁-C₁₈ alkyl)-substituted amino, mono-(C₅-C₁₈ aryl)-substituted amino, di-(C₅-C₁₈ aryl)-substituted amino, C₂-C₁₈ alkylamido (—NH—(CO)-alkyl), C₆-C₁₈ arylamido (—NH—(CO)-aryl), imino (—CR═NH where R=hydrogen, C₁-C₁₈ alkyl, C₅-C₁₈ aryl, C₆-C₁₈ alkaryl, C₆-C₁₈ aralkyl, etc.), alkylimino (—CR═N(alkyl), where R=hydrogen, C₁-C₁₈ alkyl, C₅-C₁₈ aryl, C₆-C₁₈ alkaryl, C₆-C₁₈ aralkyl, etc.), arylimino (—CR═N(aryl), where R=hydrogen, C₁-C₁₈ alkyl, C₅-C₁₈ aryl, C₆-C₁₈ alkaryl, C₆-C₁₈ aralkyl, etc.), nitro (—NO₂), nitroso (—NO), sulfo (—SO₂—OH), sulfonato (—SO₂—O—), C₁-C₁₈ alkylsulfanyl (-S-alkyl; also termed “alkylthio”), arylsulfanyl (-S-aryl; also termed “arylthio”), C₁-C₁₈ alkylsulfinyl (—(SO)-alkyl), C₅-C₁₈ arylsulfinyl (—(SO)-aryl), C₁-C₁₈ alkylsulfonyl (—SO₂-alkyl), C₅-C₁₈ arylsulfonyl (—SO₂-aryl), phosphono (—P(O)(OH)₂), phosphonato (—P(O)(O—)₂), phosphinato (—P(O)(O—)), phospho (—PO₂), and phosphino (—PH₂). Typically, hydrocarbyl moieties in the aforementioned functional groups, if acyclic, have 1 to 12 carbon atoms, while if cyclic, have 5 to 16 carbon atoms.

The aforementioned functional groups may, if a particular group permits, be further substituted with one or more additional functional groups or with one or more hydrocarbyl moieties such as those specifically enumerated above, and the term “functional group” encompasses all such instances.

When the term “substituted” appears prior to a list of possible substituted groups, it is intended that the term apply to every member of that group. For example, the phrase “substituted alkyl, alkenyl, and aryl” is to be interpreted as “substituted alkyl, substituted alkenyl, and substituted aryl.” Analogously, when the term “heteroatom-containing” appears prior to a list of possible heteroatom-containing groups, it is intended that the term apply to every member of that group. For example, the phrase “heteroatom-containing alkyl, alkenyl, and aryl” is to be interpreted as “heteroatom-containing alkyl, substituted alkenyl, and substituted aryl.”

An “analog” of a compound is one that has a structure similar to that of the compound but differs in one or more respects. For instance, a compound and an analog thereof may differ with respect to an atom, a substituent (which may or may not be a functional group), or a molecular segment. An analog may or may not be a “derivative” of the compound; it may be derived from the compound or it may be independently synthesized from one or more different reactants. An “analog” may be a “homologue” of a compound, meaning that the compound and the analog differ by a repeating unit (e.g., a methylene group).

Some of the compounds described herein may contain one or more asymmetric centers and give rise to enantiomers, diastereomers, or other stereoisomeric forms. Such a compound may be in the form of a single stereoisomer, i.e., be “stereoisomerically pure,” or contained in a mixture of two or more stereoisomers, e.g., two diastereomers, two enantiomers, or a mixture of two diastereomers and two enantiomers.

However, Δ⁹-THCV and analogs thereof, as referred to herein and unless otherwise specified, are in the following configuration (benzopyran numbering convention included):

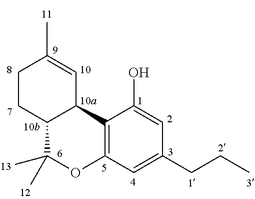 [SMILES: CCCc1cc(O)c2c(c1)OC(C)(C)[C@@H]1CCC(C)=C[C@@H]21]

### II. The Synthetic Method

The novel synthetic method results in a reaction product that comprises a Δ⁹ cannabinoid having the structure of formula (II)

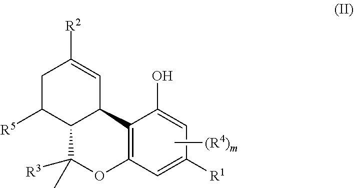

wherein:
R¹ is selected from C₁-C₁₂ hydrocarbyl, substituted C₁-C₁₂ hydrocarbyl, heteroatom-containing C₁-C₁₂ hydrocarbyl, and substituted heteroatom-containing C₁-C₁₂ hydrocarbyl,
R², R³, and R⁵ are independently selected from C₁-C₆ alkyl and substituted C₁-C₆ alkyl;
m is zero, 1 or 2; and
R⁴ is OH or OR⁶ wherein R⁶ is H, C₁-C₆ alkyl, Cs-C₁₂ aryl, or a hydroxyl protecting group, with the proviso that when m is 2, the R⁴ may be the same or different.

The synthesis uses, as a starting material, a cannabinoid reactant having the structure of formula (I)

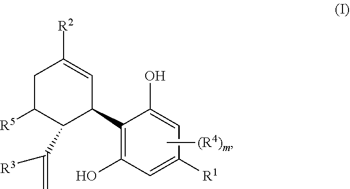

wherein all substituents are defined as for compound (II), and is carried out using reaction parameters and reaction conditions that result in a reaction product in which compound (II) predominates relative to incidentally produced by-products, particularly Δ⁸ isomers of (II).

In some embodiments, the synthetic method of the invention comprises:
(a) combining the cannabinoid reactant of formula (I) with an acid in a solvent for the cannabinoid reactant and the acid to provide a reaction mixture, wherein the acid comprises (i) a Lewis acid having an acid softness index value in the range of −10 ΔG⁰_{f, M}ⁿ⁺ to −150 ΔG⁰_{f, M}ⁿ⁺, (ii) a Brønsted acid having a pKa in the range of −4.0 to +4.0, or (iii) a combination of (i) and (ii), under reaction conditions comprising a reaction temperature in the range of −0° C. to 25° C. and a reaction time in the range of 1 hour to 24 hours;
(b) quenching the reaction mixture of (a) with a base; and
(c) removing the solvent to provide a reaction product comprising the Δ⁹ cannabinoid having the structure of formula (II).

The cannabinoid reactant of formula (I) may be obtained from a natural product, i.e.,

hemp, or it may be synthesized in whole or in part. For example, the cannabinoid reactant may be chemically synthesized using the methodology described in applicant's published international patent application WO 2022/133332 A2 and in co-pending U.S. patent application Ser. No. 18/212,061, filed Jun. 20, 2023 and published on Nov. 9, 2023 as US 2023/0357177 A1, previously incorporated by reference herein. When the cannabinoid reactant is CBDV per se (i.e., the compound of formula (I) wherein R¹ is n-propyl, R² and R³ are methyl), m is zero, and R⁵ is H), a representative synthesis thereof comprises contacting divarinol with Δ⁹-2,8-menthadien-1-ol in the presence of a Lewis acid catalyst under reaction conditions effective to result in a reaction product comprising cannabidivarin. Another representative synthesis of the CBDV reactant starts with phloroglucinol and comprises: contacting phloroglucinol with a hydroxyl-protecting reagent to provide hydroxyl-protected phloroglucinol; effecting a cross-coupling reaction of the hydroxyl-protected phloroglucinol with a reactant M-CH₂CH₂CH₃ in the presence of a catalyst that facilitates the cross-coupling reaction, wherein M comprises a metallic element, to provide hydroxyl-protected divarinol; deprotecting the hydroxyl-protected divarinol; and contacting the divarinol with Δ⁹-2,8-menthadien-1-ol in the presence of a Lewis acid catalyst under reaction conditions effective to result in a reaction product comprising cannabidivarin. Further detail may be found in the aforementioned patent applications.

In some embodiments, the R¹ substituent indicated in the molecular structures of formula (I) and (II) is selected from C₁-C₈ alkyl, substituted C₁-C₈ alkyl, heteroatom-containing C₁-C₈ alkyl, substituted heteroatom-containing C₁-C₈ alkyl, C₂-C₈ alkenyl, substituted C₂-C₈ alkenyl, heteroatom-containing C₂-C₈ alkenyl, and substituted heteroatom-containing C₂-C₈ alkenyl, while R² and R³ are C₁-C₆ alkyl and may be the same or different; R⁵ is H; and m is zero. In some embodiments, R¹ is C₁-C₈ alkyl or C₂-C₈ alkenyl. In other embodiments, R1 is C₂-C₆ alkyl. It should be noted that when R¹ is n-propyl, R² and R³ are methyl, m is zero, and R⁵ is H, the cannabinoid reactant is cannabidivarin (CBDV).

In the initial step of the reaction, the cannabinoid reactant is added to a solvent so that the concentration of the cannabinoid reactant therein is in the range of 0.25 M to 2.5 M, generally 0.25 M to 1.5 M. Any solvent or solvent combination used should dissolve the reactants and be compatible with all components of the reaction mixture without adversely affecting the intended reaction or any reactant. Representative solvents that can be used include, but are not limited to, aromatic hydrocarbons, e.g., toluene, ethyl benzene, and xylenes; alkanes such as cyclohexane; ketones, e.g., acetone, methyl ethyl ketone, diethyl ketone, methyl n-propyl ketone, acetophenone, and cyclohexanone; ethers, including linear, poly and cyclic ethers such as diethyl ether, di-n-propyl ether, di-n-butyl ether, methyl t-butyl ether, ethyl n-propyl ether, glyme, diglyme, tetrahydrofuran, 1,4-dioxane, and 1,3-dioxolane; lower (C₁-C₆) alcohols, e.g., methanol, ethanol, n-propanol, isopropanol, isomers of butanol, isomers of pentanol, ethylene glycol, propylene glycol, and glycerol; and other solvents such as dimethylformamide (DMF), dimethoxyethane (DME), dimethylacetamide (DMAC), N-methylpyrrolidone (NMP), dimethylsulfoxide (DMSO); chloroform; and dichloromethane (DCM), dichloroethane (DCE), acetonitrile (MeCN), ethyl acetate (EtOAc), and propylene carbonate.

Two or more solvents can be combined into a single solvent system, where the two or more solvents may or may not be selected from the foregoing list.

A preferred solvent is typically, although not necessarily, an aprotic, polar solvent that, in one embodiment, is non-coordinating; illustrative preferred solvents are chlorinated solvents such as DCM, DCE, and chloroform, as well as aromatic solvents such as toluene and benzene.

After combining the cannabinoid reactant and the solvent, the mixture is cooled to a temperature below 0° C., typically in the range of —25° C. to 0° C. After cooling to a reaction temperature in the aforementioned range, the selected acid is added to the reaction mixture in order to facilitate the intramolecular cyclization reaction represented in Scheme 1:

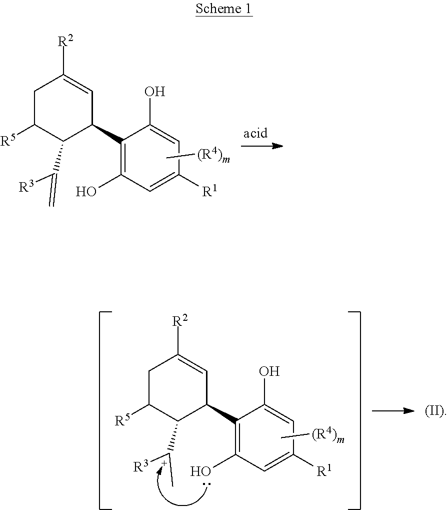

The primary parameters relied upon to select a suitable acid for catalyzing the above reaction are Lewis acid hardness and Brønsted acid pKa. Suitable Lewis acids are those having an Acid Softness Index in the range of −10 ΔG⁰_{f, M}ⁿ⁺ to −150 ΔG⁰_{f, M}ⁿ⁺, while suitable Brønsted acids are those having a pka in the range of −4.0 to +4.0, e.g., −2.0 to +2.0. A combination of two or more acids may also be used, e.g., two or more Lewis acids, two or more Brønsted acids, or a combination of at least one Lewis acid with at least one Brønsted acid.

Suitable Lewis acids are generally selected from the following: a salt of a Group 13 element of the periodic table (also referred to as Group IIIB) such as Al, B, Ga, In, and Tl; a salt of a transition metal in the +2 or +3 oxidation state, such as Zn, Ti, Mn, Fe²⁺, and Fe³⁺; or a salt of an actinide or lanthanide. Halide salts, e.g., chloride salts, may be preferred in some embodiments. By way of illustration, representative Lewis acids that can be advantageously used in the present method include AlCl₃, BCl₃, GaCl₃, InCl₃, ZnCl₂, TiCl₄, MnCl₂, FeCl₂, FeCl₃, LaCl₃, and AcCl₃.

Suitable Brønsted acids for use herein, as noted above, are those having a pKa in the range of −4.0 to +4.0, e.g., −2.0 to +2.0, insofar as acids with pKa value above these ranges may not be strong enough to promote isomerization (see the mechanism illustrated in Scheme 1), while acids with a pka value below these ranges may lead to over-isomerization, in turn resulting in generation of a larger fraction of Δ⁸ isomers in the reaction product. Suitable Brønsted acids herein include, without limitation, sulfonic acids such as p-toluenesulfonic acid (tosic acid), methanesulfonic acid, ethanesulfonic acid, 1-hexanesulfonic acid, trifluoromethanesulfonic acid, benzenesulfonic acid, 4-ethylbenzenesulfonic acid, 1,5-naphthalenesulfonic acid, and camphorsulfonic acid; carboxylic acids such as trichloroacetic acid, trifluoroacetic acid, oxalic acid, fumaric acid, phthalic acid, and formic acid; inorganic acids such as hydrochloric acid, sulfuric acid, nitric acid, and phosphoric acid; and acidic resins.

Representative Brønsted acids and their pKa values are set forth in Table 1:

TABLE 1

| Acid | pKa |
|---|---|
| p-Toluenesulfonic acid | −2.8 |
| Methanesulfonic acid | −2.0 |
| Benzenesulfonic acid | −0.60 |
| Trifluoroacetic acid | −0.3 |
| Trichloroacetic acid | 0.65 |
| Camphorsulfonic acid | 1.2 |
| Ethanesulfonic acid | 1.83 |
| 2-Nitrobenzoic acid | 2.17 |
| 2-Hydroxybenzoic acid | 2.98 |
| 1-Hexanesulfonic acid | 3.6 |
| Formic acid | 3.75 |

The relative quantities of the cannabinoid reactant and the acid are, like other reaction parameters discussed herein, selected to maximize the relative amount of the desired Δ⁹ isomer in the reaction product, i.e., the amount relative to the less desirable Δ⁸. Generally, the acid is present in the reaction mixture at a molar ratio in the range of 0.01:1 to 0.2:1 relative to the cannabinoid reactant (i.e., 1 mol % to 20 mol %), including a range of 0.05:1 to 0.1:1 (i.e., 5 mol % to 10 mol %), and a range of 0.07:1 to 0.1:1 (i.e., 7 mol % to 10 mol %).

After addition of the selected acid to the cooled cannabinoid reactant/solvent mixture, the resulting reaction mixture is stirred for an additional 1 to 24 hours, typically, although not necessarily, for an additional 1 to 8 hours, with, optionally, an increase in temperature partway through (e.g., stirring at −25° C. for 1.5 hours followed by stirring at 0° C. for an additional 4.5 hours; see Example 1). Higher temperatures should be avoided, insofar as higher temperatures lead to a precipitous drop in the Δ⁹:Δ⁸. At this point, the reaction is presumed to be complete and the reaction mixture is quenched with a suitable base, e.g., sodium bicarbonate. The solvent layer is removed using conventional means such as evaporation under vacuum, resulting in generation of the crude reaction product that comprises the desired Δ⁹ isomer having the structure of formula (II). Temperatures greater than 70° C. should be avoided in the solvent removal step; optimally, solvent removal should be carried out at a temperature of at most 50° C.

It will be understood that in some embodiments, the cannabinoid reactant has the structure of formula (I-A) while the desired reaction product, i.e., a Δ⁹ cannabinoid encompassed by the generic structure of formula (II), has the structure of formula (II-D)

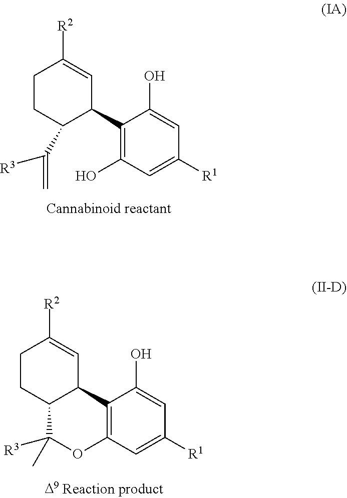

wherein, in some embodiments, R¹ is C₁-C₈ alkyl or C₂-C₈ alkenyl, e.g., C₂-C₆ alkyl, such as n-propyl; and R² and R³ are C₁-C₆ alkyl and may be the same or different.

In this embodiment, the reaction product comprises, in addition to the desired reaction product (II-D), incidentally produced by-products, i.e., the Δ⁸ isomers of (II-D), having the structures of formulae (II-A), (II-B), and (II-C), as follows:

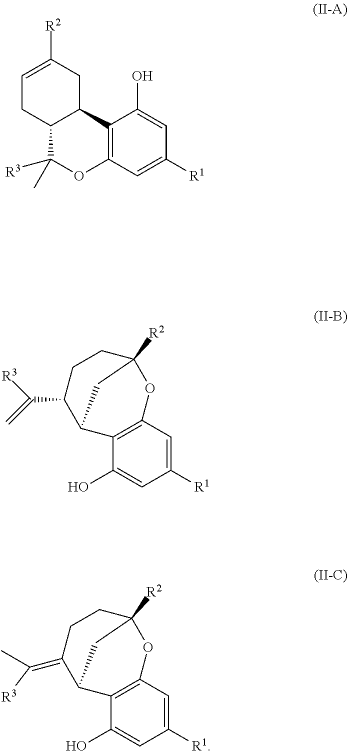

In one representative embodiment, R¹ is n-propyl and R² and R³ are methyl, such that the cannabinoid reactant is cannabidivarin (CBDV) and the desired reaction product is Δ⁹-tetrahydrocannabivarin (Δ⁹-THCV)

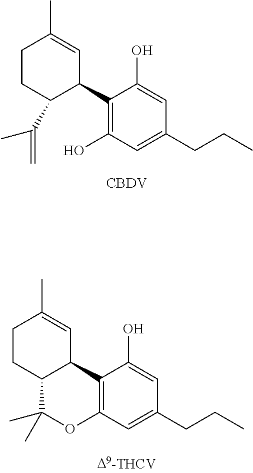

while the Δ⁸ isomers of formulae (II-A), (II-B), and (II-C) are Δ⁸-THCV, Δ⁸-iso-THCV, and Δ⁴⁽⁸⁾-iso-THCV:

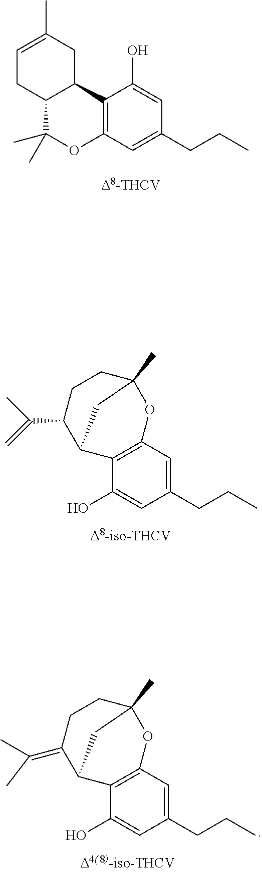

The above-described synthetic method reduces the relative quantity of the less desired Δ⁸ isomers (compounds II-A, II-B, and II-C, e.g., Δ⁸-THCV, Δ⁸-iso-THCV, and A4 (8)-iso-THCV, respectively) in the reaction product, i.e., relative to the desired Δ⁹ isomer (compound II, e.g., compound II-D, exemplified by Δ⁹-THCV). That is, the present method results in a reaction product in which the desired Δ⁹ cannabinoid having the structure of formula (II) is in a molar ratio, relative to the Δ⁸ isomers of formulae (II-A), (II-B), and (II-C), of greater than 4:1. In some embodiments, the molar ratio of the Δ⁹ cannabinoid to the Δ⁸ isomers in the reaction product is in the range of 4:1 to 50:1. In some embodiments, the molar ratio of the Δ⁹ cannabinoid to the Δ⁸ isomers in the reaction product is in the range of 9:1 to 18:1.

The aforementioned molar ratios obtained are for the crude reaction product that results from the final step of the reaction, solvent removal. Purification of the crude reaction product can result in significantly higher molar ratios of the desired Δ⁹ cannabinoid to the Δ⁸ isomers, on the order of 50:1 or greater, e.g., in the range of 50:1 to 1000:1 or greater.

Following completion of synthesis and generation of compound (I) as the desired reaction product, compound (I) may be modified, if desired, to provide further analogs, using techniques known to those of ordinary skill in the art, described in the pertinent literature and texts, or developed hereinafter.

### III. Purification of the Reaction Product

The reaction product obtained using the method of the preceding section can be purified so as to enrich the fraction of the desired Δ⁹ cannabinoid therein and provide a purified reaction product composition in which significantly higher molar ratios of the Δ⁹ cannabinoid product to the Δ⁸ isomers are achieved. It will be appreciated that various purification techniques can be implemented to achieve the desired result, and will be known to those of ordinary skill in the art or inferred from the pertinent literature. A preferred method, however, is described in Example 4, infra. The method comprises chromatographically purifying the reaction product using normal phase silica column chromatography and a simple isocratic mobile phase, e.g., a mixture of non-polar and polar organic solvents where the non-polar solvent may be heptane, hexane, petroleum ether, toluene, or the like, and polar solvent may be acetone, ethyl acetate, dichloromethane, methyl-t-butyl ether, or the like. One representative solvent combination, as documented in the Examples herein, is a mixture of heptane and acetone. Purification of gram to kilogram quantities of synthetically generated Δ⁹ cannabinoid (e.g., Δ⁹ THCV) can be achieved using commercially available or other pre-packed silica columns, up to 3 kg in size, using bulk silica. The isocratic mobile phase enables retention of cannabinoids, removal of residual solvent, and isolation of the desired Δ⁹ cannabinoid essentially free from starting materials and minor impurities. The use of an isocratic mobile phase also facilitates recovery and reuse of the solvents used for chromatographic separation.

### IV. Cannabinoid Compositions

The invention also encompasses a novel composition of matter, a cannabinoid composition comprising Δ⁹-THCV, Δ⁸-THCV, Δ⁸-iso-THCV, A4 (8)-iso-THCV, and a Lewis acid catalyst residue, wherein the molar ratio of the Δ⁹-THCV to the total of the Δ⁸-THCV, Δ⁸-iso-THCV, and Δ⁴⁽⁸⁾-iso-THCV is greater than 4:1 (e.g., in the range of 4:1 to 50:1, such as 9:1 to 18:1) and the Lewis acid catalyst residue, e.g., aluminum or another metal deriving from the selected acid used in the reaction, represents 1-150 ppm of the composition.

In another embodiment, the invention encompasses as a novel composition of matter a purified cannabinoid composition comprising Δ⁹-THCV, Δ⁸-THCV, Δ⁸-iso-THCV, Δ⁴⁽⁸⁾-iso-THCV, and a Lewis acid catalyst residue, wherein the molar ratio of the Δ⁹-THCV to the total of the Δ⁸-THCV, Δ⁸-iso-THCV, and Δ⁴⁽⁸⁾-iso-THCV is greater than 50:1 (e.g., in the range of 50:1 to 1000:1) and the Lewis acid catalyst residue, which again may be aluminum or another metal deriving from the acid used in the reaction, represents 1-150 ppm of the composition.

It is to be understood that while the invention has been described in conjunction with a number of specific embodiments, the foregoing description as well as the examples that follow are intended to illustrate and not limit the scope of the invention.

All patents, patent publications, literature references, and other materials cited herein are incorporated by reference in their entireties.

### EXAMPLE 1

### Synthesis of Δ⁹-THCV From Cannabidivarin (CBDV)

2 kg of CBDV 2 kg of CBDV was added to a 50 L jacketed reactor. The reactor was charged with 20 L of dichloromethane (DCM), giving a CBDV concentration of 0.35M. The reaction mixture was cooled to −25° C., before AlCl₃ (6 mol % of CBDV, i.e., an AlCl3:CBDV molar ratio of 0.06:1) was added to the reactor. The mixture was stirred at 150 rm for 1.5 hours before increasing the temperature to 0° C. Stirring continued at this temperature for 4.5 hours. The reaction was quenched by the addition of sodium bicarbonate. The DCM layer was dried using sodium sulfate and evaporated under a vacuum to produce the final THCV resin.

### EXAMPLE 2

The procedure of Example 1 was followed except that the concentration of CBDV in the reaction mixture was 0.7 M.

### EXAMPLE 3

The procedure of Example 1 was followed except that amount of AlCl₃ used was increased from 6 mol % to 7 mol. %.

### Results

Analysis of the reaction product composition obtained in Examples 1 through 3 was carried out, with the results provided in Table 2:

TABLE 2

| | | | | Δ⁸-THCV | |
|---|---|---|---|---|---|
| | | | | and | |
| | Total | Total THCV | | Δ⁸-THCV | |
| | cannabinoids, | obtained, | Δ⁹-THCV, | isomers, | Δ⁹:Δ⁸ |
| Example | wt. % | wt. % | wt. % | wt. % | ratio |
| 1 | 89.48 | 83.13 | 74.60 | 8.53 | 8.74 |
| 2 | 97.15 | 85.77 | 79.76 | 6.01 | 13.24 |
| 3 | 99.9 | 98.83 | 92.99 | 5.84 | 15.92 |

In the above table, the “Δ⁸-THCV isomers” refer to Δ⁸-iso-THCV and Δ⁴⁽⁸⁾-iso-THCV.

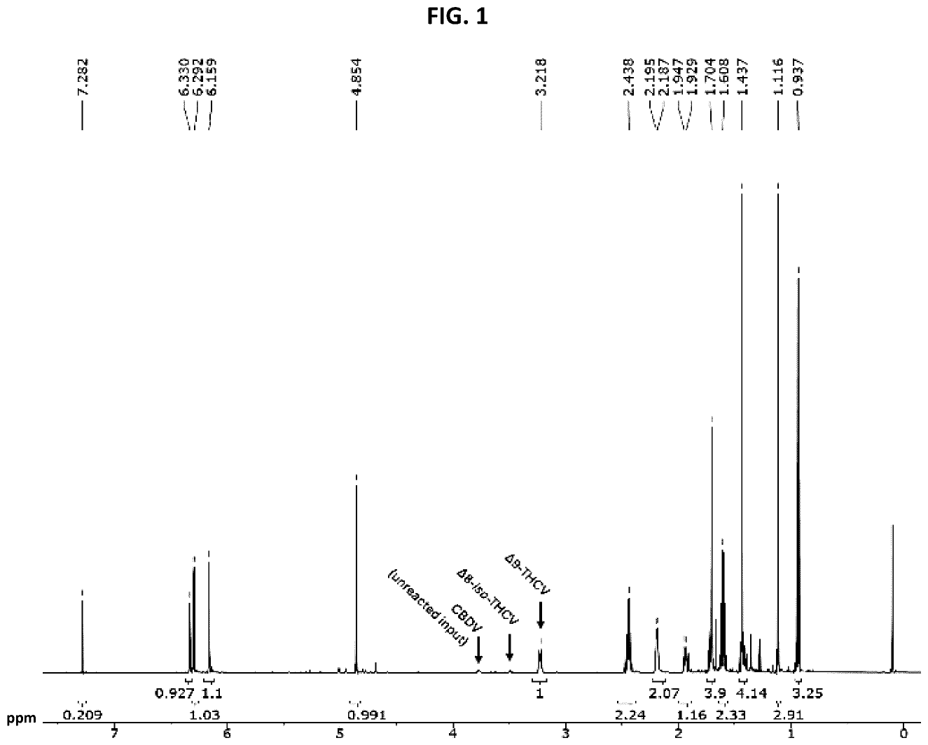

The ¹H NMR spectrum of the reaction product composition obtained in Example 3 is attached as FIG. 1 (¹H NMR (400 MHz, CDCl₃) δ 6.31 (s, 1H), 6.27 (s, 1H), 4.79 (s, 1H), 3.20 (d, J=10.79 Hz, 1 H), 2.42 (m, 2H), 2.116 (d, J =6.02 Hz, 2H), 1.92 (m, 1H), 1.68 (s, 3H), 1.58 (m, 2H), 1.42 (s,3H), 1.10 (s, 3H), 0.92 (t, J =7.29 Hz, 3H)). As indicated therein, the reaction product composition was primarily composed of Δ⁹-THCV, with several minor peaks indicating the presence of solvent, unreacted CBDV reactant, and Δ⁸-THCV isomers.

### EXAMPLE 4

### Purification of Δ⁹-THCV

The Δ⁹-THCV reaction product obtained in Example 2 was purified using normal phase silica column chromatography, as follows:

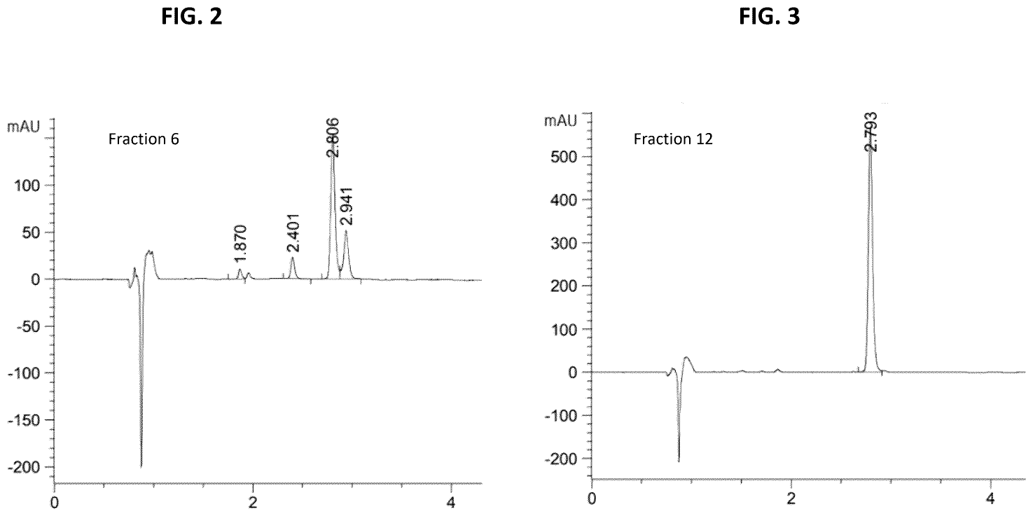

Fractions of heptane-acetone eluant were collected in an automated fashion during normal phase chromatographic separation of the D⁹-THCV reaction product synthesized as described in Example 2, using the puriFlash® ultra performance flash purification system (Advion Interchim Scientific®, Ithaca, New York). The isolated fractions were then evaporated to dryness, reconstituted in methanol, and analyzed for cannabinoid content and purity by reverse-phase high pressure liquid chromatography (HPLC). The results of the HPLC analysis demonstrated that the reactant and trace impurities were concentrated in the early eluting fractions, allowing the isolation of later eluting fractions containing highly purified D⁹-THCV. More specifically, and as illustrated in FIG. 2, HPLC analysis of Fraction 6 shows the presence of starting material (CBDV eluting at ˜1.870 min), the desired final product (D⁹-THCV eluting at ˜2.806 min), and minor impurities eluting at ˜2.401 and ˜2.941 min). In contrast, the HPLC analysis of Fraction 12, as may be seen in FIG. 3, demonstrates the presence of only one component, highly purified D⁹-THCV (eluting at ˜2.793 min).

### EXAMPLE 5

### Synthesis and Purification of CBDV Reactant

CBDV was synthesized according to Scheme 2, below.

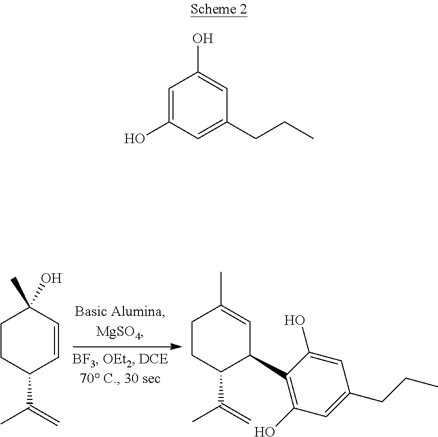 [SMILES: CCCc1cc(O)cc(O)c1 ; C=C(C)[C@H]1C=C[C@@](C)(O)CC1 ; C=C(C)[C@@H]1CCC(C)=C[C@H]1c1c(O)cc(CCC)cc1O]

To an oven-dried 5 L round bottom flask equipped with an overhead mechanical stirrer, a thermocouple, and a heating mantle, was added pre-dried divarinol (523.5 g, 3.44 mol) and 2.4 L of dry 1,2-dichloroethane. The solution was stirred for 5 minutes. Basic alumina (105.7 g, 1.04 mol) and magnesium sulfate (332.0 g, 2.76 moles) were added, and the suspension was stirred for another 5 minutes, after which BF₃·OEt₂ (13.1 mL, 0.106 mol) was added. The suspension was stirred 15 minutes under nitrogen, at room temperature.

The reaction mixture was then heated at 70° C., and a solution of (1S, 4R)-p-mentha-2,8-dien-1-ol (209.9 g, 1.37 mol) in 380 mL of anhydrous 1,2-dichloroethane was added in on portion. A slight increase of temperature was observed (˜5° C.). The mixture was stirred for 30 seconds, after which 1.2 L of a saturated aqueous solution of sodium bicarbonate was slowly added. The mixture was cooled to room temperature. The pH was measured with litmus paper to ensure that the aqueous layer was alkaline.

The entire reaction mixture was filtered on a Celite 545 pad to retrieve most of the alumina. The filtrate was inserted in an extraction funnel, and the phases were allowed to separate. The aqueous phase was extracted with 500 ml of fresh DCM. The organic phases were combined, washed with 1L of an aqueous saturated solution of sodium chloride, and dried with magnesium sulfate. After filtration on Whatman #1 filter paper, the organic fraction was evaporated to dryness, providing 754.2 g of a brown oil.

The residue was dissolved in 3.1 L of heptane and mechanically stirred for 5 minutes with a solution made of 1.7 L of methanol, 1.4 L of water, and 32 mL of an aqueous saturated solution of sodium chloride. The phases were separated using a separatory funnel. The organic layer was dried with magnesium sulfate, suction filtered on a Celite 545 pad, and evaporated to dryness.

A portion of 200 ml of fresh heptane was added to the residue and evaporated to dryness. A viscous beige solid (310.0 g) was obtained on which a portion of 550 mL of heptane was added. The heterogenous mixture was allowed to stir mechanically overnight at room temperature. It was then suction filtered on a Whatman #1 filter, providing 119.2 g of a beige powder, containing 97% of CBDV and 3% of abn CBDV, as confirmed by 1H-NMR. (The filtrate (179.4 g) contained 27% of CBDV, 61% of abn CBDV, and 12% of bis compound.)

The beige solid was recrystallized at room temperature in 1170 mL of heptane, providing 109.3 g of light-yellow needles. The heptane suspension was heated to 80° C. in order to ensure total solubility. Crystallization began at 50° C.

### EXAMPLE 6

### Synthesis and Purification of Δ⁹-THCV

This example describes synthesis of Δ⁹-THCV according to Scheme 3, below.

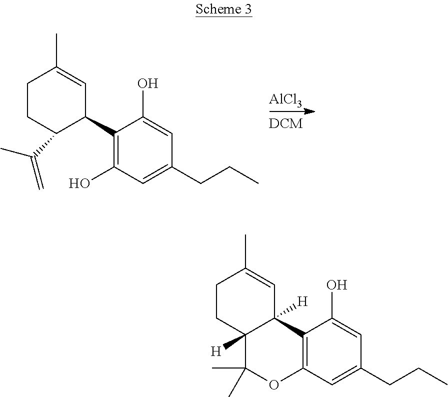 [SMILES: C=C(C)[C@@H]1CCC(C)=C[C@H]1c1c(O)cc(CCC)cc1O ; "AlCl3" ; CCCc1cc(O)c2c(c1)OC(C)(C)[C@@H]1CCC(C)=C[C@@H]21]

CBDV (1 equiv.), prepared as described in Example 5, was added to the glass reactor previously inerted to less than 1% O₂, followed by (2.7 vol.) of anhydrous DCM. The mixture was cooled to between −30 and −25° C. before gradually adding AlCl₃ (0.07 equiv.) in three equal portions with a 20-minute interval between additions, during which the reaction mixture changed from gold yellow/brown to bright red/pink/brown. After maintaining the mixture at −25° C. for an hour, the reactor was set to 0° C. and the reaction was held over five hours. Post-reaction, the mixture was washed twice with 1 volume of 10% sodium bicarbonate solution, each wash followed by settling and separation of the lower DCM layer, which was retained and the upper aqueous layer discarded. Sodium sulfate (1.17 equiv.) and basic aluminum oxide Brockman 1 (2.09 equiv.) were then added to the retained DCM layers, mixed for 1 hour, and the resulting solution was filtered using a Buchner funnel with Whatman Grade 4 cellulose filter paper (20-25 μm). The filtrate was evaporated under vacuum at 50° C. for 3-4 hours in a rotary evaporator, reconstituted in equal parts of 98% +Ethanol (1 L ethanol per kg of resin), and concentrated again to a honey-like consistency at 60° C. The final product was spread in thin layers and dried overnight at 60° C., yielding over 2.5 kg of Δ⁹-THCV, which was analyzed by HPLC-DAD, HS-GC-MS and 1H-NMR. The final Δ⁹-THCV product was a very viscous glass-like amorphous resin at room temperature, which was observed to freeze and shatter after storing at −20° C. for 6+hrs. The typical color was transparent golden yellow, but the purple color appeared rapidly when the surface contacted the non-inert atmosphere. This color change was found to have minimal effect on the quality of the product.

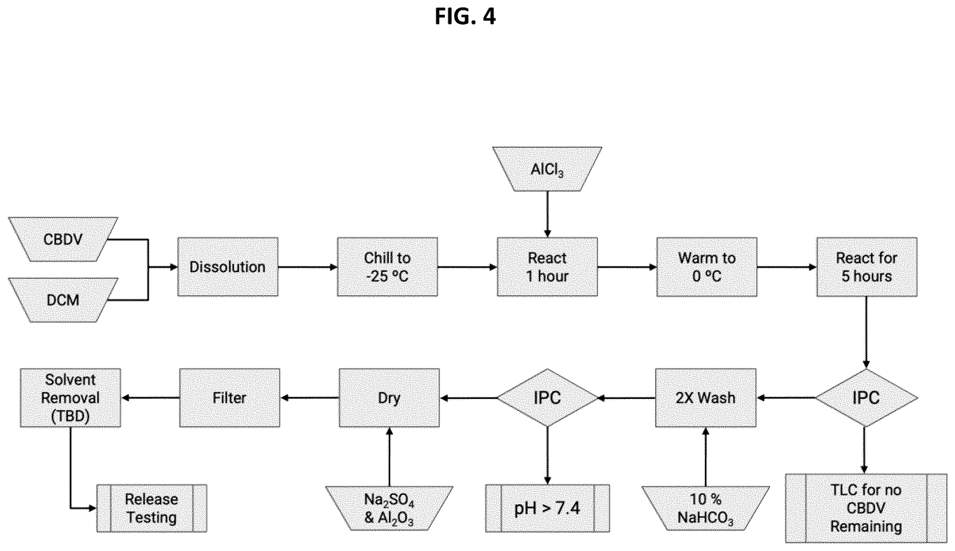

A process flow diagram for synthesis of Δ⁹-THCV is included in FIG. 4. Table 3, below, provides the results of in-process control (IPC) and release tests:

TABLE 3
In-process Control (IPC) and Release Tests

| | | Acceptable |
|---|---|---|
| Step | Test | Range |
| Reaction | Thin Layer Chromatography (TLC) | No CBDV |
| | | observed |
| Quench | pH for catalyst deactivation | pH > 7.4 |
| Release | Assay, appearance, related substances, | See detailed |
| | residual solvents, metals & bioburden | specifications |

1. A method for synthesizing a Δ⁹ cannabinoid having the structure of formula (II) 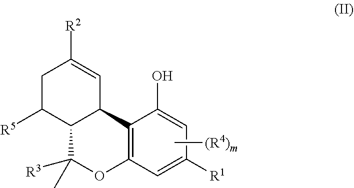
wherein:
R¹ is selected from C₁-C₁₂ hydrocarbyl, substituted C₁-C₁₂ hydrocarbyl, heteroatom-containing C₁-C₁₂ hydrocarbyl, and substituted heteroatom-containing C₁-C₁₂ hydrocarbyl,
R², R³, and R⁵ are independently selected from C₁-C₆ alkyl and substituted C₁-C₆ alkyl;
m is zero, 1 or 2; and
R⁴ is OH or OR⁶ wherein R⁶ is H, C₁-C₆ alkyl, Cs-C₁₂ aryl, or a hydroxyl protecting group, with the proviso that when m is 2, the R⁴ may be the same or different,
wherein the method comprises:
(a) combining a cannabinoid reactant having the structure of formula (I) 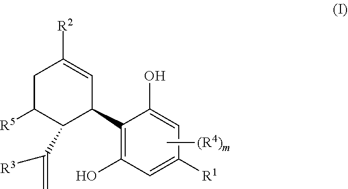
with an acid in a solvent for the cannabinoid reactant and the acid to provide a reaction mixture, wherein the acid comprises (i) a Lewis acid having an acid softness index value in the range of −10 ΔG⁰_{f, M}ⁿ⁺ to −150 ΔG⁰_{f, M}ⁿ⁺, (ii) a Brønsted acid having a pka in the range of −4.0 to +4.0, or (iii) a combination of (i) and (ii), under reaction conditions comprising a reaction temperature in the range of-0°° C. to 25°° C. and a reaction time in the range of 1 hour to 24hours;
(b) quenching the reaction mixture of (a) with a base; and
(c) removing the solvent to provide a reaction product comprising the Δ⁹ cannabinoid having the structure of formula (II).

2. The method of claim 1, wherein:
R¹ is selected from C₁-C₈ alkyl, substituted C₁-C₈ alkyl, heteroatom-containing C₁-C₈ alkyl, substituted heteroatom-containing C₁-C₈ alkyl, C₂-C₈ alkenyl, substituted C₂-C₈ alkenyl, heteroatom-containing C₂-C₈ alkenyl, and substituted heteroatom-containing C₂-C₈ alkenyl;
R² and R³ are C₁-C₆ alkyl and may be the same or different;
R⁵ is H; and
m is zero.

3. The method of claim 2, wherein the reaction product further comprises Δ⁸ isomers of the compound of formula (II), wherein the Δ⁸ isomers have the structure of formula (II-A), (II-B), and (II-C)

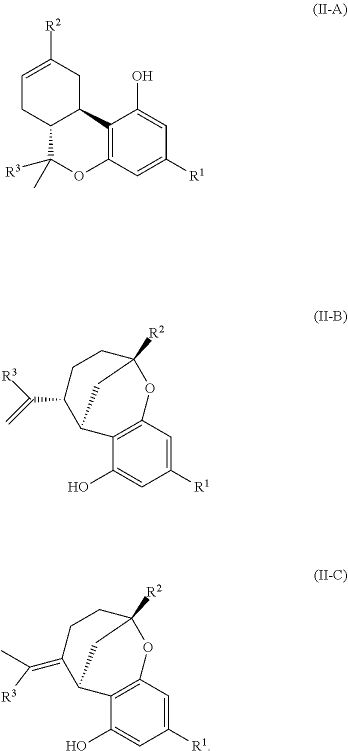

4. The method of claim 3, wherein, in the reaction product, the Δ⁹ cannabinoid having the structure of formula (II) is in a molar ratio, relative to the Δ⁸ isomers of formulae (II-A), (II-B), and (II-C), of greater than 4:1.

5. The method of claim 4, wherein the molar ratio of the Δ⁹ cannabinoid to the Δ⁸ isomers in the reaction product is in the range of 4:1 to 50:1.

6. The method of claim 5, wherein the molar ratio of the Δ⁹ cannabinoid to the Δ⁸ isomers in the reaction product is in the range of 9:1 to 18:1.

7. The method of claim 3, wherein the acid comprises a Lewis acid having an acid softness index value in the range of −10 ΔG⁰_{f, M}ⁿ⁺ to −150 ΔG⁰_{f, M}ⁿ⁺.

8. The method of claim 7, wherein the Lewis acid comprises a salt of a Group 13 cation, a transition metal in the +2 oxidation state, a transition metal in the +3 oxidation state, an actinide, or a lanthanide.

9. The method of claim 8, wherein the salt comprises a halide.

10. The method of claim 9, wherein the salt comprises a chloride selected from AlCl³, BCl³, GaCl³, InCl³, ZnCl², TiCl⁴, MnCl², FeCl², FeCl³, LaCl³, and AcCl³.

11. (canceled)

12. The method of claim 3, wherein the acid comprises a Brønsted acid having a pKa in the range of −4.0 to +4.0.

13. (canceled)

14. The method of claim 3, wherein the reaction conditions further comprise a concentration of the cannabinoid reactant in the solvent in the range of 0.25 M to 2.5 M.

15. (canceled)

16. The method of claim 3, wherein the acid is present in the reaction mixture at a molar ratio in the range of 0.01:1 to 0.2:1 relative to the cannabinoid reactant.

17. (canceled)

18. (canceled)

19. The method of claim 3, wherein:
R¹ is selected from C₁-C₈ alkyl and C₂-C₈ alkenyl, optionally substituted with (a) —(CO)—NR⁸-R⁹ wherein R⁸ and R⁹ are independently selected from H and C₁-C₁₂ hydrocarbyl, (b) —NR¹⁰—R¹¹ wherein R¹⁰ is H or C₁-C₁₂ hydrocarbyl and R¹¹ is C₆-C₁₂ hydrocarbyl, C₁-C₁₂ hydrocarbyl substituted with at least one functional group, C₁-C₁₂ heterohydrocarbyl, or C₁-C₁₂ heterohydrocarbyl substituted with at least one functional group, (c) —(SO₂)—R¹² wherein R¹² is H or C₁-C₁₂ heterohydrocarbyl, C₁-C₁₂ hydrocarbyl substituted with at least one functional group, or C₁-C₁₂ heterohydrocarbyl substituted with at least one functional group, (d) —(SO₂)—NR¹³R¹⁴ wherein R¹³ is H or C₁-C₁₂ hydrocarbyl and R¹⁴ is H or C₁-C₁₂ hydrocarbyl, 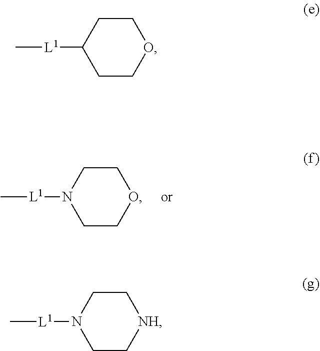
wherein L¹ is C₁-C₆ alkyl; and
R² and R³ are methyl.

20. The method of claim 19, wherein R¹ is C₁-C₈ alkyl or C₂-C₈ alkenyl.

21. (canceled)

22. The method of claim 20, wherein R¹ is n-propyl, such that the cannabinoid reactant having the structure of formula (I) is cannabidivarin and the Δ-9 cannabinoid having the structure of formula (II) is Δ⁹-tetrahydrocannabivarin.

23-26. (canceled)

27. The method of claim 3, further including purifying the reaction product composition to provide a purified reaction product.

28-30. (canceled)

31. The method of claim 27, wherein the molar ratio of the Δ⁹ cannabinoid to the Δ⁸ isomers in the purified reaction product is in the range of 50:1 to 1000:1.

32. A cannabinoid composition comprising Δ⁹-THCV, Δ⁸-THCV, Δ⁸-iso-THCV, Δ⁴⁽⁸⁾-iso-THCV, and a Lewis acid catalyst residue, wherein the molar ratio of the Δ⁹-THCV to the total of the Δ⁸-THCV, Δ⁸-iso-THCV, and Δ⁴⁽⁸⁾-iso-THCV is greater than 4:1 and the Lewis acid catalyst residue represents 1-150 ppm of the composition.

33-34. (canceled)

35. The purified cannabinoid composition of claim 32, wherein the molar ratio of the Δ⁹-THCV to the total of the Δ⁸-THCV, Δ⁸-iso-THCV, and Δ⁴⁽⁸⁾-iso-THCV is in the range of 50:1 to 1000:1.

36. (canceled)
